(19) United States
(12) Patent Application Publication
(10) Pub. No.: US 2011/0093034 A1
(43) Pub. Date: Apr. 21, 2011

(54) BIFURCATED LEAD WITH INTEGRATED ANCHOR AT BRANCH REGION

(76) Inventors: John E. Kast, Hugo, MN (US); James A. Zimmerman, Blaine, MN (US); Craig S. Pilarski, Ham Lake, MN (US); William C. Phillips, Brooklyn Park, MN (US); Thomas I. Miller, Blaine, MN (US); Mary L. Boatwright, Andover, MN (US)

(21) Appl. No.: 12/992,938

(22) PCT Filed: May 29, 2009
(86) PCT No.: PCT/US09/45579

§ 371 Date: Nov. 16, 2010

Related U.S. Application Data
(60) Provisional application No. 61/058,294, filed on Jun. 3, 2008.
(60) Provisional application No. 61/175,482, filed on May 5, 2009.

(51) Int. Cl. A61N 1/36; A61N 1/04

(52) U.S. Cl. 607/46; 607/149

(57) ABSTRACT

An implantable medical lead includes a proximal portion having first and second contacts. The lead further includes a first distal arm having a first electrode that is electrically coupled to the first contact, and includes a second distal arm having a second electrode that is electrically coupled to the second contact. The lead also includes a branch region where the proximal portion transitions to the first and second distal arms. A tissue anchoring element is attached to the branch region for securing the branch region to tissue of a patient into which the lead is implanted. Such bifurcated leads may be used to apply electrical signals to occipital nerves of the patient via the electrodes. A lead extension includes a distal connector with two lead receptacles and a tissue anchoring element attached to the connector. An adaptor having three lead receptacles includes an anchoring element attached thereto.

### FIELD

The present disclosure relates to implantable medical devices; more particularly to medical leads capable of delivering electrical signals to two discrete anatomical locations, such as a left and a right occipital nerve.

### BACKGROUND

Headaches, such as migraines, and occipital neuralgia are often incapacitating and may lead to significant consumption of drugs to treat the symptoms. However, a rather large number of people are unresponsive to drug treatment, leaving them to wait out the episode or to resort to coping mechanisms. For refractive occipital neuralgia, nerve ablation or separation may effectively treat the pain.

Occipital nerve stimulation may serve as an alternative for treatment of migraines or occipital neuralgia. For example, a dual channel implantable electrical generator may be implanted subcutaneously in a patient. A distal portion of first and second leads may be implanted in proximity to a left and right occipital nerve such that one or more electrode of the leads are in electrical communication with the occipital nerves. The proximal portions of the leads may then be connected to the signal generator such that electrical signals can be delivered from the signal generator to the electrodes to apply therapeutic signals to the occipital nerves. Alternatively, two single channel implantable electrical generators may be employed, where the first lead is connected to one signal generator, and the second lead is connected to the second signal generator. In either case, the lead is typically tunneled subcutaneously from site of implantation of the signal generator to the occipital nerve or around the base of the skull. Such tunneling can be time consuming and is invasive.

In addition to being time consuming and invasive, tunneling through long distances of tissue or through regions, such as the neck, can lead to a good deal of strain on the lead through the patient's body movements. Such strain can result in undesired movement of the distal portion of the lead, resulting in decreased efficacy due to lead, particularly electrode, migration.

### BRIEF SUMMARY

The present disclosure, among other things, describes leads, systems and methods for applying electrical signals to occipital nerves. In some embodiments, bifurcated leads or lead extensions are described. By using bifurcated leads or extensions, only one tunneling procedure is needed to tunnel a proximal portion of a lead between a location near the occipital nerves and the implantation site of the electrical signal generator. Such leads and procedures may reduce surgery time and invasiveness associated with occipital nerve stimulation. In various embodiments, the leads are anchored to the patient's tissue at a location near the occipital nerves, via an anchoring element located at branch point of the bifurcated lead or extension. Such anchoring can greatly reduce movement of the distal portion of the lead caused by strain proximal the branch point; e.g. via movement of the neck and torso.

In an embodiment, an implantable medical lead includes a proximal portion having first and second contacts. The lead further includes a first distal arm having a first electrode that is electrically coupled to the first contact, and includes a second distal arm having a second electrode that is electrically coupled to the second contact. The lead also includes a branch region where the proximal portion transitions to the first and second distal arms. A tissue anchoring element is attached to the branch region for securing the branch region to tissue of a patient into which the lead is implanted. Such bifurcated leads may be used to apply electrical signals to occipital nerves of the patient via the electrodes.

In an embodiment, a lead extension includes a proximal portion having first and second contacts. The extension further includes a connector having first and second lead receptacles. The first lead receptacle has an internal contact electrically coupled with the first contact of the proximal portion. The second lead receptacle has an internal contact electrically coupled with the second contact of the proximal portion. The extension also includes a tissue anchoring element attached to the connector for securing the connector to tissue of a patient in which the extension is implanted. Such an extension may be employed to deliver electrical signals, via leads inserted into the receptacles of the connector, to occipital nerves of the patient.

In an embodiment, described is an adaptor for coupling a first lead configured to operably couple with an active implantable medical device with second and third leads configured to carry an electrical signal to or from tissue of a patient. The adaptor includes a body forming a first, second and thirds openings for receiving the first, second and third leads. The adaptor further includes first, second and third lead receptacles contiguous with the first, second and third openings, respectively. The first lead receptacle is configured to receive the first lead and has first and second internal contacts. The second lead receptacle is configured to receive the second lead and has an internal contact electrically coupled to the first internal contact of the first receptacle. The third lead receptacle is configured to receive the third lead and has an internal contact electrically coupled to the second internal contact of the first receptacle. The adaptor further includes an anchoring element attached to the body member. Such anchors may be used in therapies in which electrical signals are delivered to occipital nerves of a patient.

The leads, extensions, signal generators, systems and methods described herein provide one or more advantages over prior leads, extensions, signal generators, systems and methods. Such advantages will be readily understood from the following detailed description when read in conjunction with the accompanying drawings.

### BRIEF DESCRIPTION OF THE DRAWINGS

FIG. 1 is a schematic side view of an implantable system including a signal generator, lead extension and lead.

FIGS. 2A-B are schematic diagrams showing distal portions of bifurcated leads implanted in subjects and positioned to apply an electrical signal to left and right occipital nerves.

FIGS. 3 and 4A are schematic side views of a representative bifurcated leads having an anchoring element attached to a branch region.

FIGS. 4B-D are schematic cross-sections of alternative embodiments of the proximal portion of the lead shown in FIG. 4A taken through line 4b-4b.

FIG. 4E is a schematic side view of an embodiment of the branch region of the lead depicted in FIG. 4A, showing conductors running through the branch region.

FIGS. 5-6 are schematic side views of representative bifurcated leads.

FIGS. 7-8 are schematic side views of lead extensions having bifurcating connectors and associated leads.

FIG. 9 is a schematic cross section of a representative connector of a lead extension.

FIG. 10A is a schematic side view of an adaptor having receptacles for receiving leads.

FIG. 10B is a schematic cross-sectional view of an embodiment of the adaptor of FIG. 10A showing the receptacles.

FIGS. 11A-E are schematic side views of representative bifurcated leads having extensible portions.

FIGS. 12A-F are schematic side views of representative bifurcated leads having attached anchors.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”.

“Exemplary” or “representative” is used herein in the sense of “for example” or “for purposes of illustration”, and not in a limiting sense.

As used herein, “attached”, as it relates to a tissue anchoring element and a branch region of a lead or extension, means to affix the anchoring element to the branch region. The anchoring element is affixed or attached to the branch region prior to implantation; e.g. during manufacture of the lead. For example, the anchoring element may be fastened to, adhered to, secured to, integrally formed with, etc. the branch region. In various embodiments, the anchoring element is permanently attached to the branch region.

The present disclosure, among other things, describes leads, systems and methods for applying electrical stimulation signals to occipital nerves. In some embodiments, bifurcated leads or lead extensions are described. By using bifurcated leads or extensions, only one tunneling procedure is needed to tunnel a proximal portion of a lead between a location near the therapeutic site of action, such as the occipital nerves, and the implantation site of the electrical signal generator. Such leads or extensions and procedures may reduce surgery time and invasiveness associated with implant procedures, such as those associated with occipital nerve stimulation. In various embodiments, the leads or extensions are anchored to the patient's tissue at a location near the occipital nerves, via an anchoring element located at branch point of the bifurcated lead or extension. Such anchoring can greatly reduce movement of the distal portion of the lead cause by strain proximal the branch point; e.g. via movement of the neck and torso.

Nearly any implantable medical device or system employing leads may be used in conjunction with the bifurcating leads, extensions or adaptors described herein. Representative examples of such implantable medical devices include hearing implants, cochlear implants; sensing or monitoring devices; signal generators such as cardiac pacemakers or defibrillators, neurostimulators (such as spinal cord stimulators, brain or deep brain stimulators, peripheral nerve stimulators, vagal nerve stimulators, occipital nerve stimulators, subcutaneous stimulators, etc.), gastric stimulators; or the like. For purposes of occipital nerve stimulation, electrical signal generators such as Medtronic, Inc.'s Restore® or Synergy® series of implantable neurostimulators may be employed.

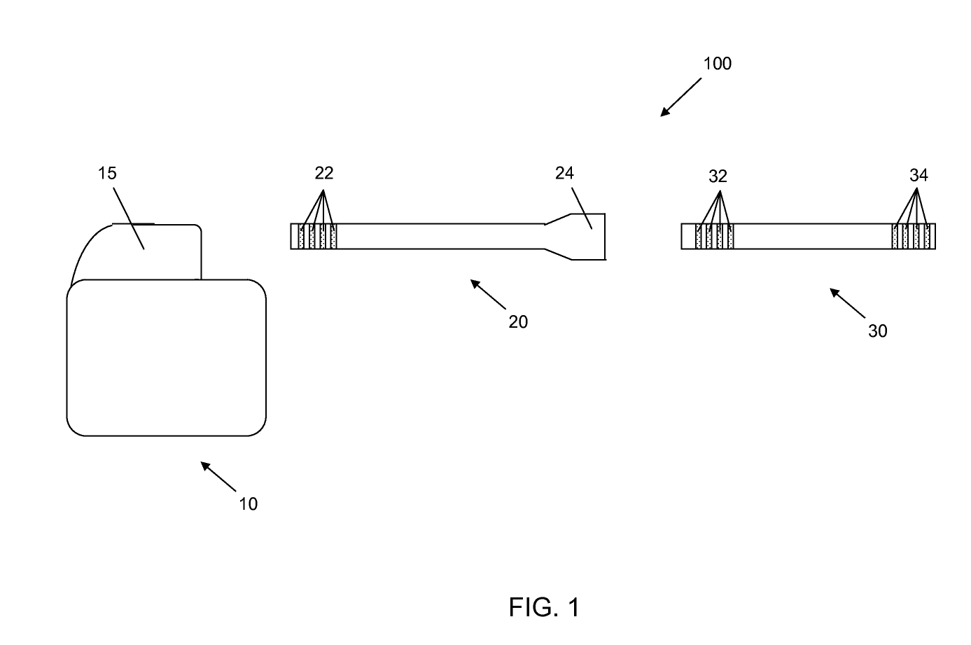

Referring to FIG. 1, a schematic side view of a representative electrical signal generator system 100 is shown. In the depicted system 100, the electrical signal generator 10 includes a connector header 15 configured to receive a proximal portion of lead extension 20. The proximal portion of lead extension 20 contains a plurality of electrical contacts 22 that are electrically coupled to internal contacts (not shown) at distal connector 24 of lead extension 20. The connector header 15 of the signal generator 10 contains internal contacts (not shown) and is configured to receive the proximal portion of the lead extension 20 such that the internal contacts of the connector header 15 may be electrically coupled to the contacts 22 of the lead extension 20 when the lead extension 20 in inserted into the header 15.

The system depicted in FIG. 1 further includes a lead 30. The depicted lead 30 has a proximal portion that includes a plurality of contacts 32 and a distal portion that includes a plurality of electrodes 34. Each of the electrodes 34 may be electrically coupled to a discrete contact 32. The distal connector 24 of the lead extension 20 is configured to receive the proximal portion of the lead 30 such that the contacts 32 of the lead 30 may be electrically coupled to the internal contacts of the connector 24 of the extension 20. Accordingly, a signal generated by the signal generator 10 may be transmitted to a patient by an electrode 34 of lead 30 when lead is connected to extension 20 and extension 20 is connected to signal generator 10.

It will be understood that lead 30 may be coupled to signal generator 10 without use of an extension 20. Any number of leads 30 or extensions 20 may be coupled to signal generator 10. Typically, one or two leads 30 or extensions 20 are coupled to signal generator 10. While lead 30 is depicted as having four electrodes 34, it will be understood that lead 30 may include any number of electrodes 34, e.g. one, two, three, four, five, six, seven, eight, sixteen, thirty-two, or sixty-four. Corresponding changes in the number of contacts 32 in lead 30, contacts 22 and internal contacts in connector 24 of lead extension, or internal contacts in connector 15 of signal generator 10 may be required or desired.

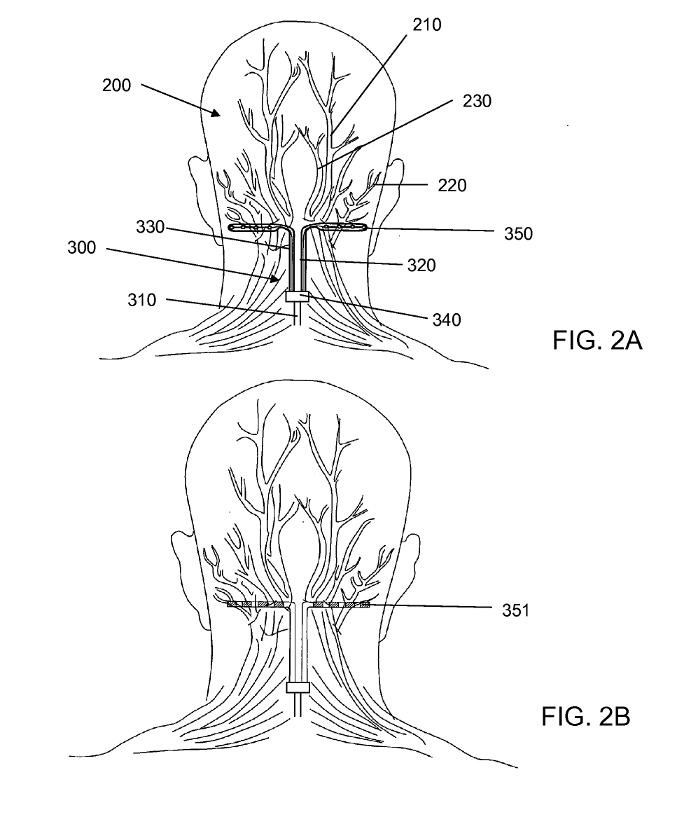

Referring to FIGS. 2A-B, bifurcated leads 300 are shown implanted in a patient to provide bilateral therapy to left and right occipital nerves 200. As used herein, occipital nerve 200 includes the greater occipital nerve 210, the lesser occipital nerve 220 and the third occipital nerve 230. The greater and lesser occipital nerves are spinal nerves arising between the second and third cervical vertebrae (not shown). The third occipital nerve arises between the third and fourth cervical vertebrae. The portion of the occipital nerve 200 to which an electrical signal is to be applied may vary depending on the disease to be treated and associated symptoms or the stimulation parameters to be applied. In various embodiments, the lead distal portions 350, 351 that contain electrodes are placed to allow bilateral application of electrical signals to the occipital nerve 200 at a level of about C1 to about C2 or at a level in proximity to the base of the skull. The position of the electrode(s) may vary. It will be understood that the electrode need not, and in various embodiments preferably does not, contact the nerve to apply the signal to the nerve. It will be further understood that a signal may be applied to any suitable portion of an occipital nerve, whether at a trunk, branch, or the like. In various embodiments, one or more electrodes are placed between about 1 cm and about 8 cm from the midline to effectively provide an electrical signal to the occipital nerve 200. Of course, the leads, adaptors, and extensions described herein may be employed for delivering therapy to any suitable location other than, or in addition to, occipital nerves. However, for purposes of illustration, simplicity and clarity, therapy will be described herein mainly with regard to occipital nerve stimulation.

As shown in FIG. 2A, a bifurcated lead 300 may include a paddle shaped distal portion 350 containing electrodes. Such paddle shaped leads are often referred to as surgical leads. Examples of surgical leads that may be used or modified to form leads as described herein include Medtronic Inc.'s Resume, SymMix, On-Point, or Specify series of leads. Surgical leads typically contain electrodes that are exposed through one face of the paddle, providing directional stimulation. The depicted bifurcated lead 300 also includes a single proximal portion 310 that allows for only one tunneling procedure to the signal generator (not shown) implant site. In addition, the bifurcated lead 300 contains a branch region 340 and first 320 and second 330 distal arms. As shown in FIG. 2B, the bifurcated lead may include distal portion 351 that include electrodes that are generally cylindrically shaped. Such leads are often referred to percutaneous leads. Examples of percutaneous leads that may be used or modified to form leads as described herein include Medtronic Inc.'s Quad Plus, Pisces Quad, Pisces Quad Compact, or 1×8 SubCompact, 1×8 Compact, and 1×8 Standard leads. Such percutaneous leads typically contain ring electrodes that apply an electrical stimulation signal to tissue in all directions around the ring. Accordingly, the amplitude of the signal (and thus the energy required from the signal generator) applied may be greater with percutaneous leads that surgical leads for occipital nerve therapies.

While distal portions 350, 351 of the leads are shown in FIGS. 2A-B as being positioned horizontally, it will be understood that the distal portions 350, 351 of the leads may be placed vertically, in a V-shaped pattern, or the like. The desired relative orientation of the distal portions 350, 351 of the leads may vary from patient to patient to achieve proper therapeutic effect, from implanting physician to implanting physician based on implant technique used, or the like.

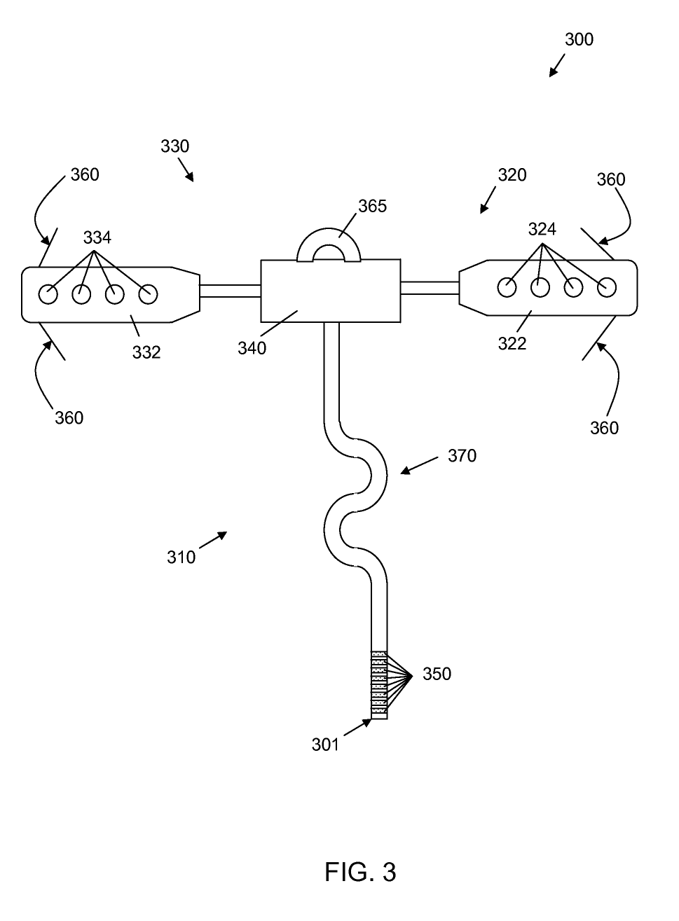

Referring now to FIG. 3, a bifurcated lead 300 includes an anchoring element 365 attached to the branch region 340. In the depicted embodiment, the anchoring element 365 includes a suture loop, but any other suitable anchoring mechanism, such as tines, may be employed. While only one anchoring element 365 or suture loop is shown, it will be understood that more than one anchoring element 365 may be attached to the branch region 340.

For application of therapies to an occipital nerve, where proximal portion 310 is tunneled through the neck region of a subject, it may be desirable to securely anchor branch region 340 to tissue of the subject to prevent movement of the lead (and thus proximal portion 310) from causing movement of distal arms 320, 330 or portions thereof It may further be desirable to secure the branch region 340 in a location of the patient where movement of the patient results in minimal movement of the lead 300 at that location relative to the patient. In some embodiments, the branch region 340 is secured via anchoring element 365 in the patient's neck, near the midline. For example, the branch region 340 may be secured near the base of the skull. If the branch region 340 is anchored near the middle of the neck or near the base of the skull, there should be little strain placed on the lead 300 distal the branch region 340, because a lead implanted in a patient from the neck midline, near the base of the skull, to a location adjacent an occipital nerve would not significantly move relative to the patient when the patient moves their head or neck. Anchoring the branch point 340 in such a location not only serves to minimize strain placed on the lead 300 proximal the branch region 340 from transferring to the distal arms 320, 330 of the leads, but also serves to reduce strain at the distal arms 320, 330 of the leads that might be experienced if the branch region 340 were secured at a tissue location subjected to movement stresses.

In addition to securing the lead 300 to the patient at the branch region 340 via anchoring element 365, it may be desirable for proximal portion 310 to contain an extensible portion 370. In the embodiment depicted in FIG. 3, the extensible portion 370 is a sigma- or serpentine-shaped portion. However, it will be understood that extensible portion 370 may be formed of and constructed from any suitable material(s) that allow for extension to reduce strain at the proximal portion 310 of the lead 300 from being transferred to the branch region 340. Such materials, including resilient polymers, and construction, including coiled conductors, are well known and may readily be employed to make a lead extensible as described herein. The extensible portion 370 may occupy any portion of or all of the length of the proximal portion 310 between the proximal end 301 and the branch region 340. The extensible region 370 serves as a strain relief feature to prevent stretching and movement of the neck (and thus proximal portion 310) from transferring excessive force to branch region 340. Of course, strain relief may be achieved via implant procedure. For example, one or more portions of the lead may be looped within the patient during the implant procedure to provide strain relief.

As further depicted in FIG. 3, the lead 300 includes contacts 350 disposed at the proximal portion 310, and electrodes 324, 334 disposed at paddle shaped portion 322, 332 of the distal arms 320, 330. Conductors (not shown) disposed within lead body electrically couple discrete contacts 350 to discrete electrodes 324, 334. As further shown, the lead 300 may include one or more anchoring elements 360, such as tines, suture loops, or the like, at the first 320 or second 330 distal arms or to portions thereof, such as the distal portions containing electrodes as depicted. In some embodiments, the anchoring elements 360 are integrally formed with the distal arms 320, 330 of the lead 300.

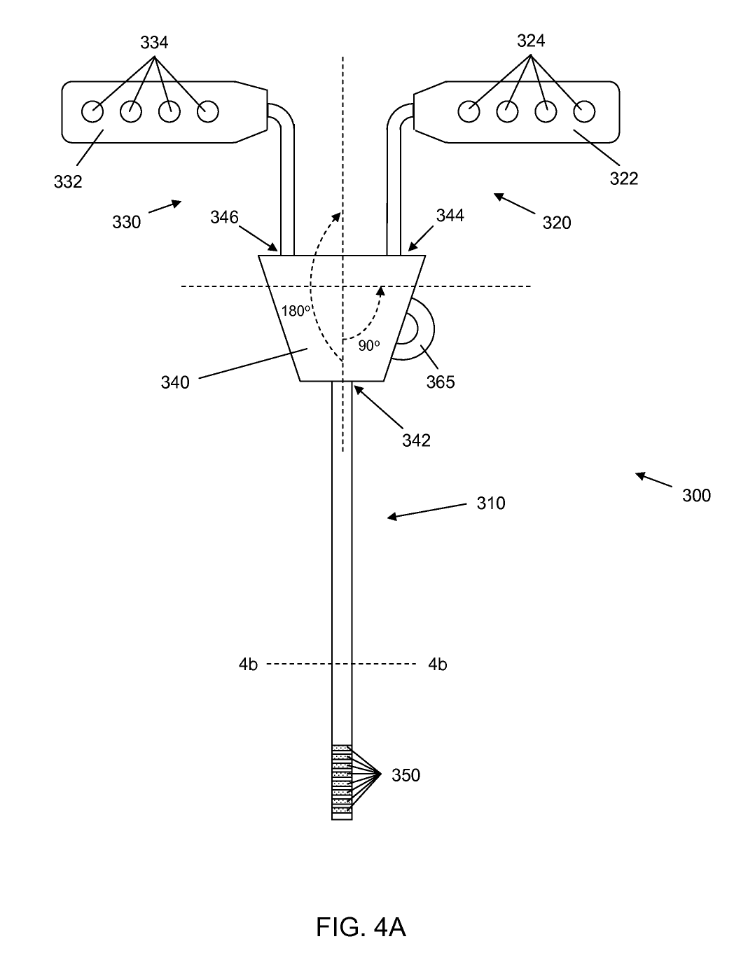

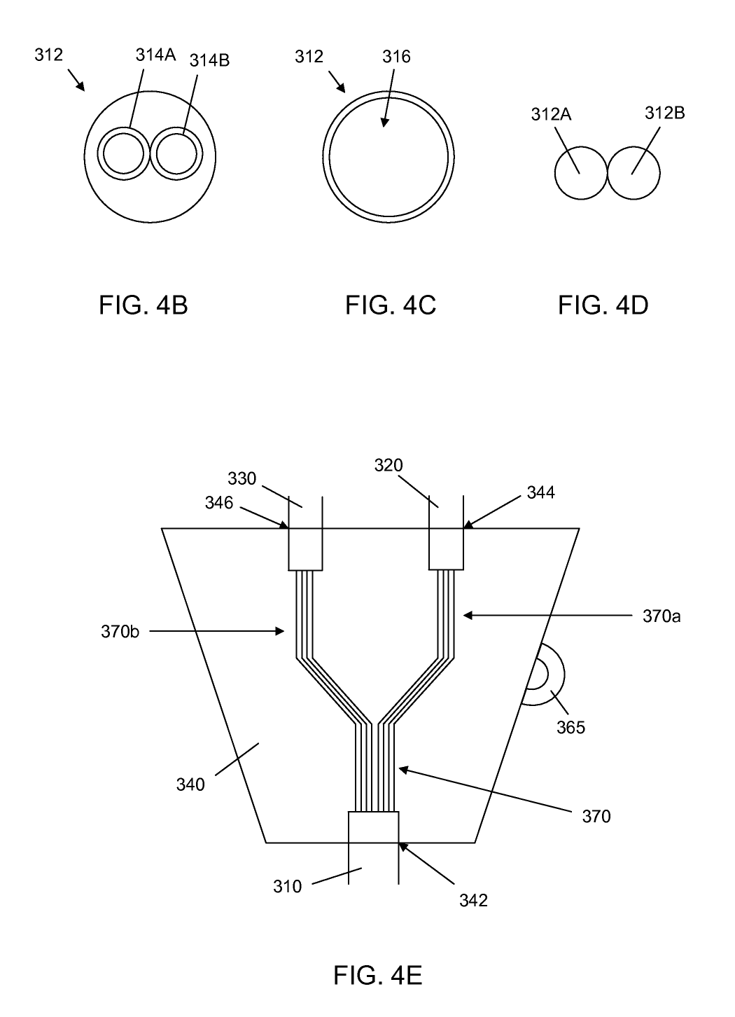

Referring now to FIG. 4A, a schematic side view of a representative bifurcated lead 300 is shown. As with the lead depicted in FIG. 3, the lead 300 depicted in FIG. 4A includes an anchoring element 365 attached to the branch region 340. The lead 300 depicted in FIG. 4A also includes a proximal portion 310 that includes a plurality of contacts 350 for electrically coupling to an electrical signal generator or a lead extension or an adaptor. The lead also includes first 320 and second 330 distal arms that contain electrodes 324, 334. The electrodes 324, 334 are electrically coupled to contacts 350 via conductors that run within lead 300 from the contacts 350 to the electrodes 324, 334. The lead 300 further includes a branch region 340 where the lead 300 transitions from the proximal portion 310 to the distal arms 320, 330. The branch region 340 may be of any suitable size and shape. In various embodiments, the branch region 340 has a volume of less than about 10 cubic centimeters.

The branch region 340 includes a first entry region 342 where the proximal portion 310 of the lead enters the branch region. The branch region 340 also includes second 344 and third 346 entry regions where the first 320 and second 330 distal arms enter the branch region. A plane runs through the centers of the entry regions 342, 344, 346. The angle of either of the second 344 and third 346 entry regions from a line extending in the plane and aligned with the geometric center first entry point 342 as it extends to proximal portion 310 of the lead 300 is between about 90 degrees and 180 degrees. In some embodiments, the center of the second 344 or third 346 entry region is substantially perpendicular to the line extending in the plane and aligned with the geometric center first entry point 342 (see, e.g., FIG. 3). In some embodiments, the angle of the second 344 or third 346 entry region relative to the first entry point 342 is between about 110 degrees and about 160 degrees.

Referring now to FIG. 4B-D, which is a cross section of the proximal portion 310 of the lead 300 depicted in FIG. 4A taken along line 4b-4b, showing representative configurations. As shown in FIG. 4B, the proximal portion of the lead includes a lead body 312. The lead body 312 may include two lumens or tubes 314A, 314B (or any number of tubes or lumens, e.g. one for each conductor) through which or around which conductors (not shown) may run to connect proximal contacts with electrodes of the first and second distal arms. Of course, the lumens or tubes 314A, 314B may be solid and the conductors can run in separate tracks along the length of the proximal portion of the lead until connecting with the distal arms. Alternatively, as shown in FIG. 4C, the lead body 312 in the proximal portion may include a single lumen 316 or solid core (not shown) and the conductors (not shown) may run in a single track along the along the length of the proximal portion of the lead. Alternatively, as shown in FIG. 4D, the proximal portion of the lead may include two attached lead bodies 312A, 312B through which separate channels of conductors (not shown) run. Of course, the lead body of the proximal portion of lead body may be configured in any other suitable manner.

Referring now to FIG. 4E, a representative example of a branch region 340 is shown in which the branch region 340 is transparent for purposes of illustration. In the depicted embodiment, a set of conductors 370 exit a lead body from the proximal portion 310 of the lead. The set of conductors 370 are separated into subsets 370a, 370b that independently enter lead bodies of the first 320 and second 330 distal arms. Any suitable manner of forming branch region 340 and separating conductors 370 for entry of subsets 370a, 370b into distal arms 320, 330 may be employed. For example, a lead body containing conductors 370 in proximal portion 310 may be formed. Additional lead bodies containing conductor subsets 370a, 370b forming distal arms 320, 330 may be formed. The conductor subsets 370a, 370b may be appropriately electrically coupled to the set of conductors 370 and branch region 340 may be overmolded over conductors 370, 370a, 370b, resulting in branch region 340 as depicted. Of course, any other suitable process may be employed to form branch region 340 and appropriately electrically couple proximal portion 310 of the lead to the distal arms 320, 330.

In the embodiment depicted in FIG. 4, the anchoring element 365 is integrally formed with the branch region 340. However, it will be understood that the anchoring element 365 may be attached to the branch point via any suitable mechanism.

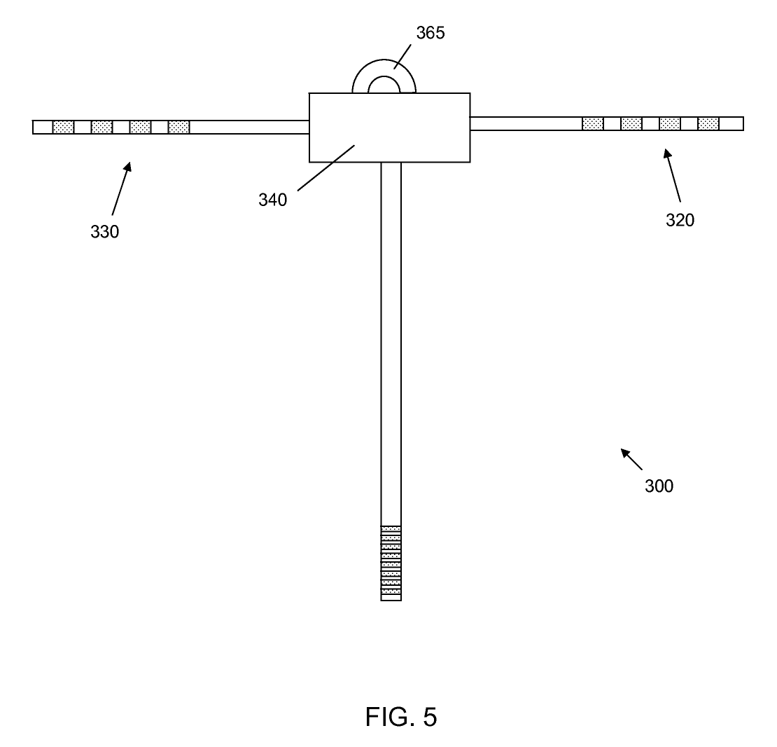

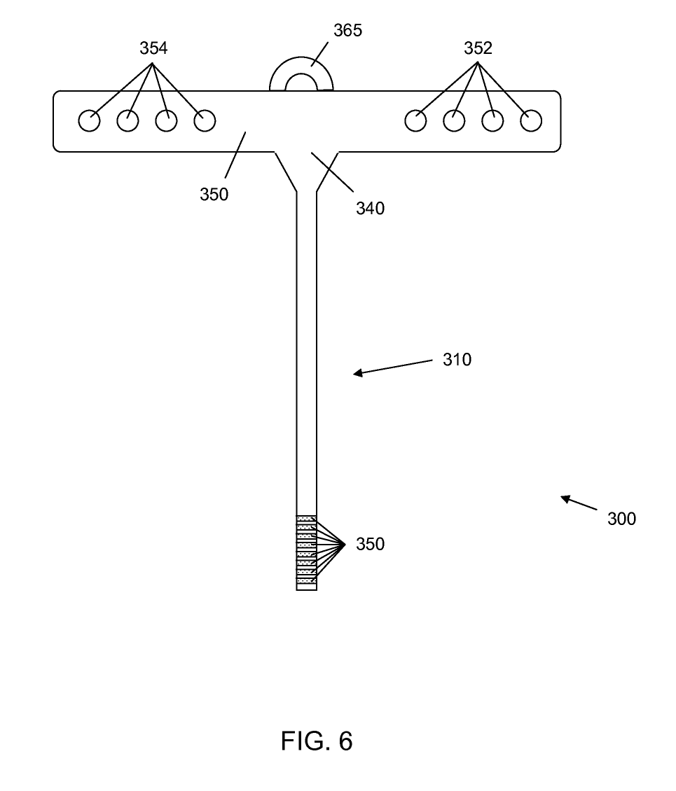

Additional representative examples of bifurcated leads 330 having anchoring elements 365 attached to a branch region 340 are depicted in FIGS. 5-6. In the embodiment depicted in FIG. 5, the portions of the distal arms 320, 330 containing the electrodes are substantially cylindrical. Of course, distal portions containing the electrodes may have any suitable shape.

Referring now to FIG. 6, a side view of a representative lead 300 is shown. The lead includes a proximal portion 310 containing contacts 350 and a distal portion 350 substantially perpendicular to the proximal portion 310. The distal portion 350 includes first 352 and second 354 sets of electrodes that are electrically coupled to the contacts 350. The first 352 and second 354 sets of electrodes are spaced apart from one another. In the embodiment depicted, the distal portion 350 can be considered to include two arms with one being to one side of the midline of the proximal portion 310 and the other being to the other side of the midline.

The leads presented in FIGS. 3-6 are intended to show that a bifurcated lead having a branch region with an attached anchoring element may take nearly any suitable form.

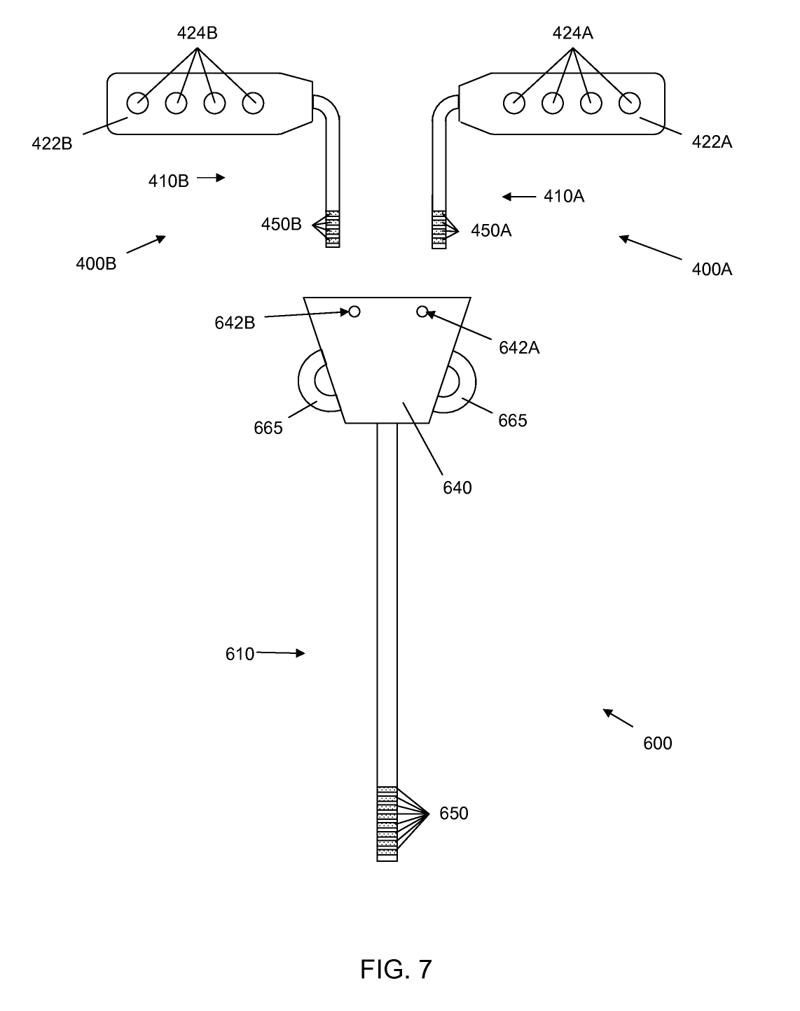

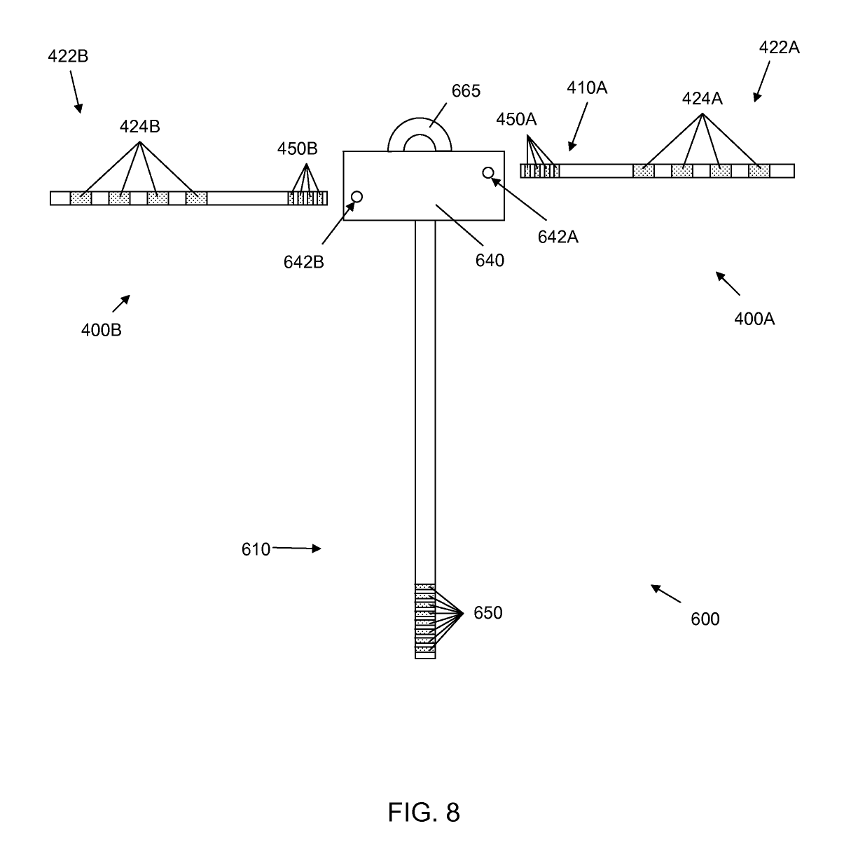

Referring now to FIGS. 7-8, examples of bifurcating lead extensions 600 are shown. The depicted lead extensions 600 include connector region 640 to provide for coupling of leads 400A, 400B to the extension 600. One or more anchoring elements 665 are attached to the connector 640. Bifurcating lead extensions 600 as described herein have many of the advantages discussed above with regard to bifurcating leads. For example, only one tunneling procedure is needed to proximal portion 610 of extension 600 to the site of implantation of signal generator. In addition, anchoring the connector 640 to tissue of the patient into which the extension is implanted can reduce the transfer of strain experienced at the proximal portion 610 of the extension 600 to leads 400A, 400B operably coupled to the extension 600 (relative to an un-anchored connector region 640).

Still with reference to FIGS. 7-8, the proximal portion 610 of the extension 600 includes contacts 650 for electrical coupling the extension 600 to the signal generator. The distal portion of extension 600 includes a connector 640 containing two lead receptacles (not shown) having internal contacts for coupling to contacts 450A, 450B of leads 400A, 400B. The connector 600 may be of any suitable size and shape. In various embodiments, the connector 600 has a volume of less than about 10 cubic centimeters, less than about 5 cubic centimeters, or less than about 2 cubic centimeters. Set screws 642A, 642B may be used to secure leads 400A, 400B in receptacles. Of course, any other suitable mechanism for securing leads 400A, 400B in receptacles may be employed. In the embodiment depicted in FIG. 8, the lead receptacles (not shown) are generally perpendicular to the angle of entry of the proximal portion 610 into connector 640.

Leads 400A, 400B include proximal portions 410A, 410B containing contacts 450A, 450B and distal portions 422A, 422B containing electrodes 424A, 424B. By employing a bifurcating extension 600 and separate leads 400A, 400B introducer tools, such as needle introducers with lumens, may be used to position distal portion 422A, 422B of leads 400A, 400B. For bifurcating leads alternative methods for introducing distal portions may be desired.

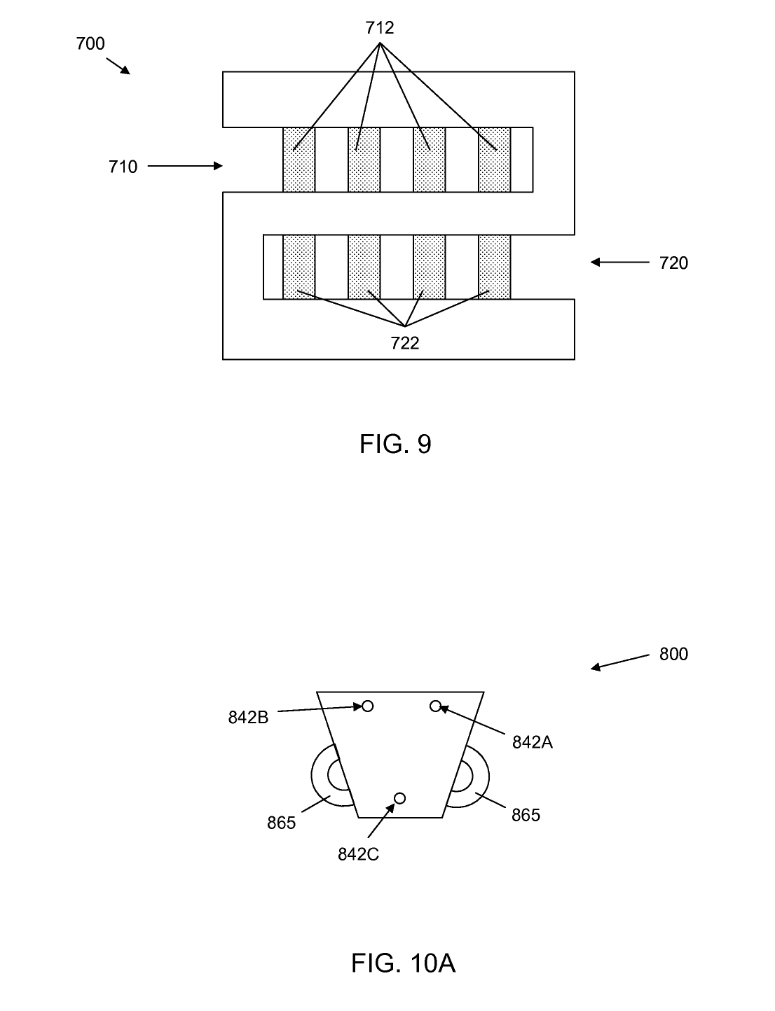

Referring now to FIG. 9, a schematic cross-section of a connector portion 700 of a lead extension; e.g. connector 640 as depicted in FIG. 8, is shown. The depicted connector 700 is shown without an attached anchoring element for the sake of simplicity. The depicted connector 700 includes first 710 and second 720 lead receptacles. The receptacles 710, 720 include openings on opposing ends of connector 700 for inserting leads in to the receptacles 710, 720 and include internal contacts 712, 722 for electrically coupling to contacts of leads when inserted into the receptacles 710, 720. The internal contacts 712, 722 of the receptacles are electrically coupled to contacts at the proximal portion of the extension via conductors (not shown). The connector 700 depicted in FIG. 9 provides for insertion of leads into the receptacles in generally opposing orientation. However, it will be understood that the connector 700 and the receptacle of the connector may be constructed in any suitable orientation, configuration, or fashion.

Regardless of the size, shape or configuration of the connector 700 of an extension, in various embodiments where the extension is employed in occipital nerve therapy, the connector is secured via one or more anchoring element in a patient's neck, near the midline. For example, the connector may be secured near the base of the skull.

In the embodiments depicted in FIGS. 7-9, the connector has a body with receptacles (not shown in FIGS. 7-8) for receiving leads. However, it will be understood that the connectors may be at distal ends of arms (e.g., if extensions took the general form of leads described with regard to FIGS. 3-6) or otherwise located or configured. In such cases, it may be desirable for an anchor to be attached to a branch point of the extension (e.g., as described with regard to the leads depicted in FIGS. 3-6).

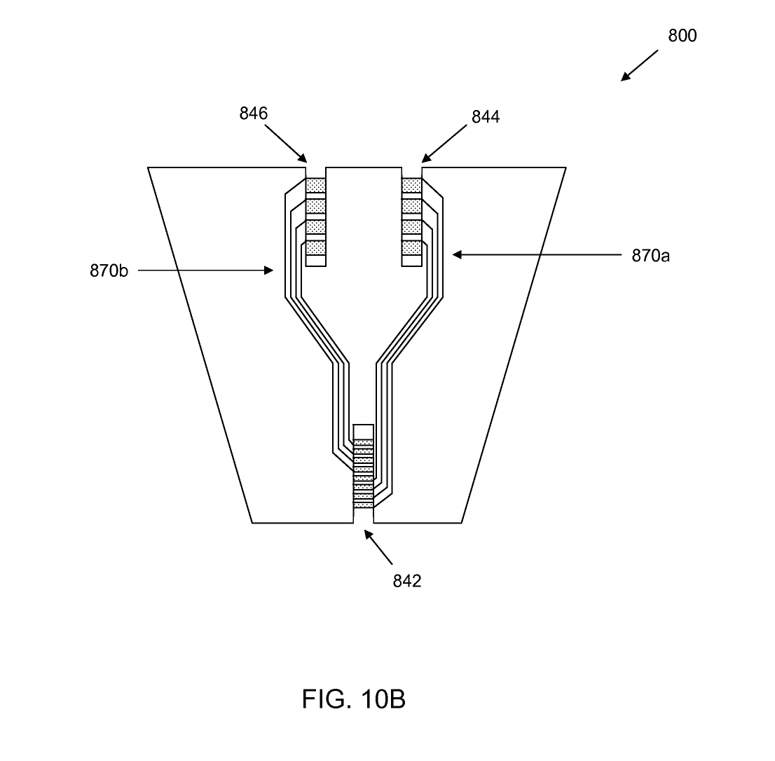

Referring now to FIG. 10A, a representative adaptor 800 that can be used to couple one incoming lead to two therapeutic leads is shown. One or more anchoring elements 865 are attached to or form a part of the adaptor 800. In the depicted embodiment, the adaptor 800 includes set screws 842A, 842B, 842C for securing leads within receptacles (not shown in FIG. 10A) of the adaptor. Of course, any other suitable mechanism may be employed to secure leads within the receptacles.

An adaptor 800 may be of any suitable size and shape. In various embodiments, the adaptor 800 has a volume of less than about 10 cubic centimeters; e.g., less than about 5 cubic centimeters. Regardless of the size, shape or configuration of the connector 700 of an extension, in various embodiments where the extension is employed in occipital nerve therapy, the connector is secured via one or more anchoring element in a patient's neck, near the midline. For example, the connector may be secured near the base of the skull.

Referring now to FIG. 10B, a schematic cross-sectional view of an embodiment of an adaptor 800 as depicted in FIG. 10A is shown. The anchoring elements are not shown in FIG. 10B for purposes of simplicity. As shown, the adaptor 800 includes a first receptacle 842 having internal contacts for electrically coupling to external contacts of a lead or extension operably coupled to an active implantable device, such as an electrical signal generator. The internal contacts of the first receptacle 842 are electrically coupled to discrete internal contacts of second 844 and third 846 receptacles of the adaptor 800. The second 844 and third 846 receptacles are configured to receive leads that are configured to carry electrical signals to or from a patient's tissue. The internal contacts of the second 844 and third 846 receptacles are configured to couple to external contacts of such leads when the leads are inserted into the receptacles. Conductors 870a, 870b electrically couple the internal contacts of the first receptacle 842 to the contacts of the second 844 and third 846 receptacles.

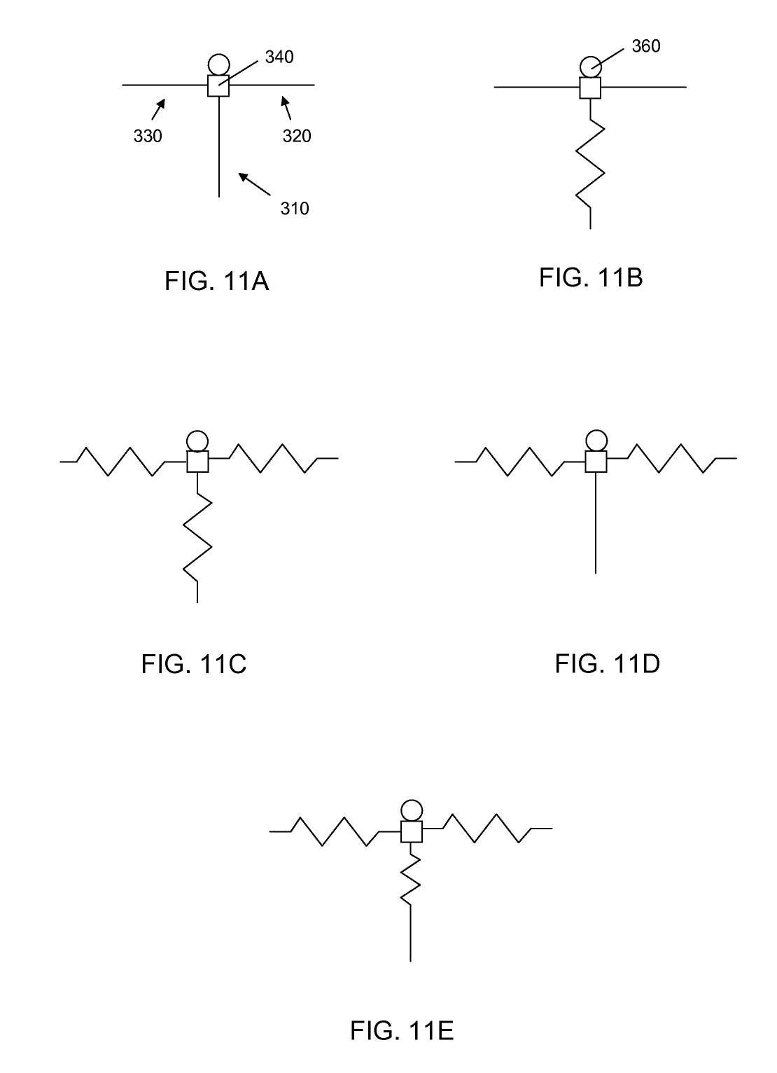

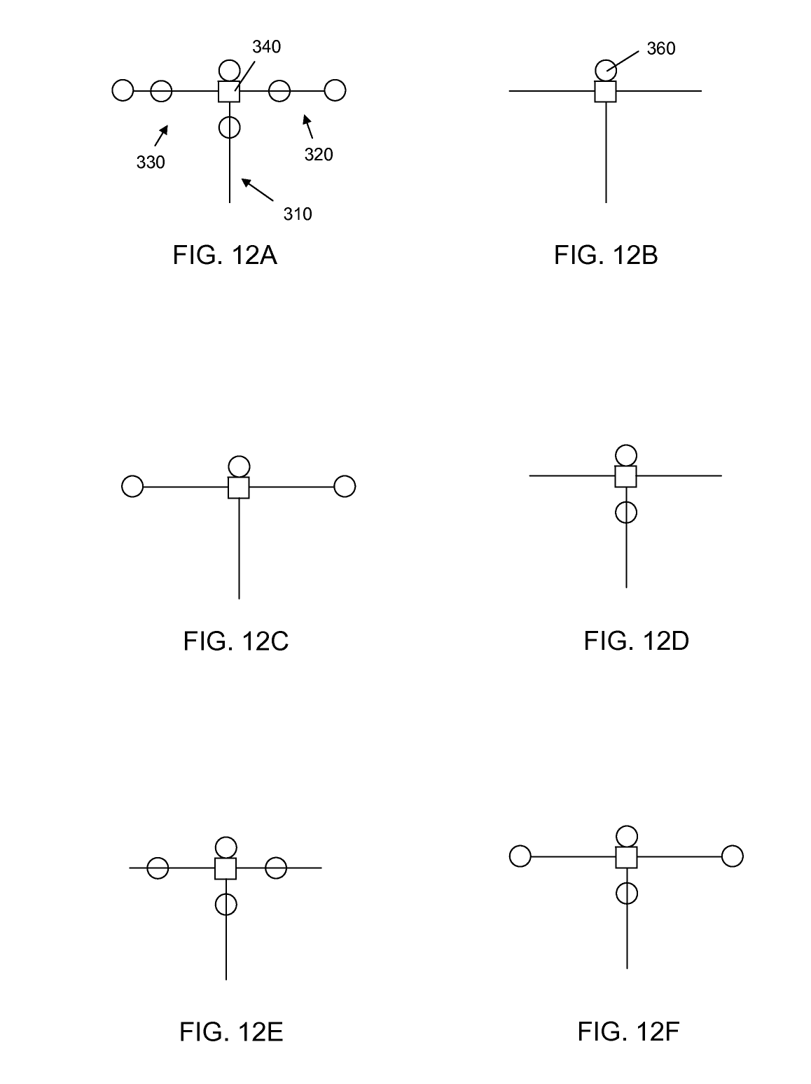

Referring now to FIGS. 11-12, various representative configurations of bifurcated leads are shown. However, it will be understood that the configurations presented may also be applied to bifurcating extensions (e.g. as described with regard to FIGS. 7-8), as appropriate. Further, while T-shaped configurations are depicted, it will be understood that such configurations are readily applicable to Y- or other shaped configurations. In the embodiments depicted in FIGS. 11A-E, the bifurcated leads include a proximal portion 310 containing contacts (not shown), a branch region 340 and first 320 and second 330 distal arms containing electrodes (not shown). The squiggly lines depicted in FIGS. 11B-E represent extensibility of the lead at the squiggly portion. Extensibility may include a sigma shaped section, loops, or may otherwise be configured to be extensible. As depicted, proximal portion 310 or distal arms 320, 330 or portions thereof may be extensible.

As shown in FIGS. 12A-F, in which circles represent attached anchors 360, a bifurcated lead may include one or more attached anchor at nearly any location of the lead, such as the distal portion or along the length of a distal arm 320, 330, at a branch region 340, or anywhere along the proximal portion 310. It will be understood that possible combinations of the configurations shown in FIGS. 11-12 are contemplated, as are combinations of other figured depicted and discussed herein.

In various embodiments described and depicted herein, anchor element (365, 666, 865) is depicted as a suture loop. However, it will be understood that any other suitable configuration, suturable or otherwise, may be employed. For example, a groove or bulbous protrusion may serve as a suitable suturable anchor element.

Thus, embodiments of BIFURCATED LEAD WITH INTEGRATED ANCHOR AT BRANCH REGION are disclosed. One skilled in the art will appreciate that the leads, extensions, connectors, devices such as signal generators, systems and methods described herein can be practiced with embodiments other than those disclosed. The disclosed embodiments are presented for purposes of illustration and not limitation.

1. An implantable medical lead comprising:
a proximal portion including a proximal end, a first contact and a second contact;
a first distal arm including a first electrode, the first electrode electrically coupled to the first contact;
a second distal arm including a second electrode, the second electrode electrically coupled to the second contact;
a branch region where the proximal portion transitions to the first and second distal arms; and
a tissue anchoring element attached to the branch region.

2. A lead according to claim 1, wherein the anchoring element is integrally formed with the branch region.

3. A lead according to claim 1, wherein the anchoring element comprises a suture loop.

4. A lead according to claim 1, wherein the lead comprises an extensible section between the proximal end and the branch region.

5. A system comprising:
a lead comprising:
a proximal portion including a proximal end, a first contact and a second contact;
a first distal arm including a first electrode, the first electrode electrically coupled to the first contact;
a second distal arm including a second electrode, the second electrode electrically coupled to the second contact;
a branch region where the proximal portion transitions to the first and second distal arms; and
a tissue anchoring element attached to the branch region; and
a lead extension comprising a lead receptacle having first and second internal contacts, wherein the first internal contact of the lead receptacle is configured to electrically couple with the first contact of the lead and wherein the second internal contact of the lead receptacle is configured to electrically couple with the second contact of the lead.

6. A system according to claim 5, further comprising an electrical signal generator operably couplable to the lead via the extension.

7. A system comprising:
a lead comprising:
a proximal portion including a proximal end, a first contact and a second contact;
a first distal arm including a first electrode, the first electrode electrically coupled to the first contact;
a second distal arm including a second electrode, the second electrode electrically coupled to the second contact;
a branch region where the proximal portion transitions to the first and second distal arms; and
a tissue anchoring element attached to the branch region; and
an electrical signal generator comprising a lead receptacle having first and second internal contacts, wherein the first internal contact of the lead receptacle is configured to electrically couple with the first contact of the lead and wherein the second internal contact of the lead receptacle is configured to electrically couple with the second contact of the lead.

8. A method for applying an electrical signal to left and right occipital nerves of a subject, the method comprising:
implanting a lead, in the subject, the lead comprising:
a proximal portion including a proximal end, a first contact and a second contact;
a first distal arm including a first electrode, the first electrode electrically coupled to the first contact;
a second distal arm including a second electrode, the second electrode electrically coupled to the second contact;
a branch region where the proximal portion transitions to the first and second distal arms; and
a tissue anchoring element attached to the branch region;
wherein the first electrode of the first distal arm is positioned so that it is capable of applying an electrical signal to the left occipital nerve and wherein the second electrode of the second distal arm is positioned so that it is capable of applying an electrical signal to the right occipital nerve;
anchoring, via the anchoring element, the branch region to tissue of the subject;
applying a first electrical signal to the left occipital nerve via the first electrode; and
applying a second electrical signal to the right occipital nerve via the second electrode, wherein the first and second electrical signals are the same or different.

9. A method according to claim 8, further comprising:
tunneling the proximal portion of the lead to a site of implantation of an implantable electrical signal generator; and
electrically coupling the lead to the signal generator.

10. A lead extension comprising:
a proximal portion having a proximal end and including first and second contacts;
a distal portion including a connector, the connector having first and second lead receptacles, the first lead receptacle having a first internal contact electrically coupled with the first contact of the proximal portion, the second lead receptacle having a second internal contact electrically coupled with the second contact of the proximal portion; and
a tissue anchoring element attached to the connector.

11. A lead extension according to claim 10, wherein the tissue anchoring element is integrally formed with the branch region.

12. A lead extension according to claim 10, wherein the tissue anchoring element comprises a suture loop.

13. A lead extension according to claim 10, wherein the lead extension comprises an extensible section between the proximal end and the branch region.

14. A system comprising:
a lead extension comprising:
a proximal portion having a proximal end and including first and second contacts;
a distal portion including a connector, the connector having first and second lead receptacles, the first lead receptacle having a first internal contact electrically coupled with the first contact of the proximal portion, the second lead receptacle having a second internal contact electrically coupled with the second contact of the proximal portion; and
a tissue anchoring element attached to the connector; and
an electrical signal generator comprising a receptacle having first and second internal contacts, wherein the first internal contact of the receptacle is configured to electrically couple to the first contact of the proximal portion of the lead extension and wherein the second internal contact of the lead receptacle is configured to electrically couple with the second contact of the proximal portion of the lead extension.

15. A method for applying an electrical signal to left and right occipital nerves of a subject, the method comprising:
implanting a lead extension in the subject, the lead extension comprising:
a proximal portion having a proximal end and including first and second contacts;
a distal portion including a connector, the connector having first and second lead receptacles, the first lead receptacle having a first internal contact electrically coupled with the first contact of the proximal portion, the second lead receptacle having a second internal contact electrically coupled with the second contact of the proximal portion; and
a tissue anchoring element attached to the connector;
implanting in the subject a first lead having a first contact and a first electrode electrically coupled to the first contact, wherein the first electrode is positioned so that it is capable of applying an electrical signal to the left occipital nerve;
implanting in the subject a second lead having a second contact and a second electrode electrically coupled to the second contact, wherein the second electrode is positioned so that it is capable of applying an electrical signal to the right occipital nerve;
electrically coupling the first contact of the first lead to the first internal contact of the first receptacle of the lead extension;
electrically coupling the second contact of the second lead to the second internal contact of the second receptacle of the lead extension;
anchoring, via the tissue anchoring element, the connector of the lead extension to tissue of the subject;
applying a first electrical signal to the left occipital nerve via the first electrode; and
applying a second electrical signal to the right occipital nerve via the second electrode, wherein the first and second electrical signals are the same or different.

16. A method according to claim 15, further comprising:
tunneling the proximal portion of the lead extension to a site of implantation of an implantable electrical signal generator; and
electrically coupling the lead extension to the signal generator.

17. An adaptor for coupling a first lead configured to operably couple with an active implantable medical device with second and third leads configured to carry an electrical signal to or from tissue of a patient, the adaptor comprising:
a body forming a first, second and thirds openings for receiving the first, second and third leads;
a first lead receptacle contiguous with the first opening and configured to receive the first lead, the first receptacle having a first and second internal contacts;
a second lead receptacle contiguous with the second opening and configured to receive the second lead, the second receptacle having an internal contact electrically coupled to the first internal contact of the first receptacle;
a third lead receptacle contiguous with the third opening and configured to receive the third lead, the third receptacle having an internal contact electrically coupled to the second internal contact of the first receptacle; and
an anchoring element attached to the body member.
